(19) United States
(12) Patent Application Publication
(10) Pub. No.: US 2011/0200667 A1
(43) Pub. Date: Aug. 18, 2011

(54) VETERINARY PHARMACEUTICAL FORMULACION THAT COMPRISES AN RNA RECOMBINANT PARTICLE THAT ENCODES FOR A CU/ZN SUPEROXIDE DISMUTASE PROTEIN OF RUMINANT PATHOGENIC BACTERIA AND AT LEAST ONE RNA ALPHAVIRUS BELONGING TO THE SEMLIKI FOREST VIRUS FAMILY

(75) Inventors: Angel Oñate Contreras, Concepcion (CL); Edilia Andrews Garcia, Concepcion (CL); Gabriel Donoso Ñanculao, Concepcion (CL)

(73) Assignee: UNIVERSIDAD DE CONCEPCION, Concepcion, Santiago (CL)

(21) Appl. No.: 13/057,620

(22) PCT Filed: Aug. 7, 2009
(86) PCT No.: PCT/CL2009/000009

§ 371 Date: Apr. 28, 2011

(30) Foreign Application Priority Data
Aug. 7, 2008 (CL) 2322-2008

(51) Int. Cl. A61K 9/127; A61K 31/7088; A61P 31/04

(52) U.S. Cl. 424/450; 514/44 R

(57) ABSTRACT

The technology is a veterinary pharmaceutical formulation of two vaccines, one from an RNA viral vector system constituted by an RNA recombinant particle that codifies for a Cu/Zn superoxide dismutase protein of Brucella abortus, and the other based on naked RNA constituted by a recombinant molecule of naked RNA that carries a sequence for the synthesis of at least one recombinant Cu/Zn superoxide dismutase protein of Brucella abortus and some Semliki Forest virus genes. An expression system based on the Semliki Forest virus and a use of this system, in addition to a method for the preparation of the pharmaceutical formulations.

This technology is designed for the stockbreeding sector, specifically for bovines, which have a high rate of abortions caused by the bacterium Brucella abortus.

### Previous Techniques

Brucella abortus is a facultative, intracellular gram-negative bacterium that contains mannose molecules facilitating adherence to the mononuclear phagocytes of the host. In particular, the bovine placenta contains a great number of mannose receptors, which is favor the internalization of this bacterium and consequently the probability of abortions in these animals

When the macrophage recognizes conservation patterns on the surface of Brucella sp. (LPS or external membrane proteins) it is activated and then phagocytizes the bacterium. However, Brucella sp. survives very efficiently within the phagocytic cell, since it is able to avoid the fusion of the lysosome with the phagosome. Brucella sp. avoids the respiratory burst inside the phagolysosome, since it avoids the formation of oxygen derived radicals, and in addition releases cell products such as RNA, which inhibit some lysosomal enzymes

The proteins Cu—Zn superoxide dismutase and catalase are periplasmic enzymes that detoxify the superoxide ion (O₂⁻) and hydrogen peroxide (H₂O₂), produced by the phagocytes after phagocytosis of the bacterium. The expression of these enzymes favors the continued presence of Brucella sp. inside the viable phagocyte.

The most effective immune response developed by the host against Brucella sp., is the secretion of antibodies and the activation of T lymphocytes. These are produced specifically against this bacterium; however, as this is an intracellular bacterium, the cellular immune response is the most important one in the eradication of the microorganism from the host.

Development of Vaccines Against Brucella abortus

A series of prophylactic vaccines have been developed for preventive purposes, the majority of which use attenuated bacterial strains or antigenic components specific to the bacterium (LPS and proteins of the bacterium); very few use expression vectors that encode bacterial antigenic proteins.

Among the strains mostly commonly used in attenuated vaccines is the vaccine whose active component uses strain 19 of Brucella abortus, but this vaccine causes abortions in the immunized animal and also develops antibodies against the O antigen of the LPS, interfering with the serological diagnosis of this disease. Another formulation is produced with strain 45/20 (rough strain), which, although it does not interfere with the serological diagnosis, can revert to its virulent smooth form. A third vaccine uses the strain RB51 of Brucella abortus, which is a natural mutant of the strain 2308 of Brucella abortus, whose principal characteristic is the lack of the O antigen of the LPS, for which reason it does not interfere with the serological diagnosis either. One important aspect is of the B. abortus strain 2308, is that it produces placentitis in impregnated cows and may revert to its virulent form.

The use of LPS as the active component for a possible vaccine has been tested, but it was observed that it offered no protection against Brucella sp.

Immunization with recombinant proteins has been investigated with great interest as Brucella sp. possesses a large quantity of proteins capable of inducing some type of immune response in the host.

In addition, immunization with plasmid expression vectors is a recent technique, with which encouraging results have been obtained in injectable pharmaceutical compositions, protection levels being similar to those obtained with the use of the attenuated strain of Brucella abortus RB51. The advantage of this methodology in relation to vaccination with attenuated strains lies in its easy handling and in its capacity to generate prolonged immune responses, with a high biosecurity level. However, the possibility exists that the plasmid DNA may be incorporated into the cell genome, for which reason its future use remains in doubt

In addition to plasmid vectors, there are other expression vectors, such as those based on the Semliki Forest virus (SFV). This RNA alphavirus presents a 42S RNA region that encodes a polyprotein called viral replicase, responsible for RNA genome replication, which is used as a mold for the synthesis of subgenomic 26S RNA and of viral RNA genome. Subgenomic 26S RNA encodes for the structural proteins, which correspond to Capsid proteins (C). When recently synthesized, these can unite with one or more encapsidation sequences of viral genomic RNA.

These vectors may consist basically of self-replicable naked RNA, whose sequence contains an insert of the gene of interest, which encodes for the protein with immune capacity, or for suicidal viral particles of the Semliki Forest virus, which contain RNA without a replicative capacity. Previous experiments have demonstrated the great efficiency of these systems in the production of heterologous proteins in eucariot cells, as well as the capacity to confer high levels of protection in immunized animals, even surpassing traditional DNA vaccines.

Taking into consideration the efficiency of these expression vectors, in addition to the demonstrated immune capacity of the Cu/Zn superoxide dismutase (SOD) protein, which confers protection against Brucella abortus, two SOD protein expression systems have been invented, one based on the Semliki Forest virus and the other on RNA particles. Both are capable of providing protection against this bacterium.

There are a number of documents that describe inventions related in some way to the present initiative. The most relevant documents are discussed in the following section:

European invention patent application EP1108433A3 describes a vaccine against brucellosis and the combined use of an antigenic protein “r”, in addition to a polysaccharide type “A” or “M”. The technology applied includes the use of structural components of different bacteria that can express these antigens. No part of the patent mentions the possible use of the SOD protein.

US invention U.S. Pat. No. 6,264,952 describes another type of vaccine whose active component is a bacterial agent (Brucella sp.). This bacterium is irradiated with gamma emissions, resulting in a bacterium that is metabolically active but which cannot replicate itself, so that it does not interfere with the invention being claimed.

British invention patent application GB2227936 describes an improved vaccine against Brucella abortus that makes it possible to identify cattle infected with other field strains. To this end, a combination of the main proteins of B. abortus are used as specific antigens. This immunizing agent is a pathogenic strain that can take various forms, such as purified proteins of the aforementioned bacterium, or dead or attenuated bacteria. Synthetic peptides with antigenic epitopes, obtained from the same bacteria, are another type of immunizing agent: for example, Omps I, II, Ill and the envelope protein of 7 and 8 kD. Other agents are crude or pure recombinant extracts from transformed E. coli, for the expression of the same proteins, or crude or pure recombinant extracts from transformed E. coli for the expression of the same live modified B. abortus proteins, the DNA being selected that encodes for one or more of these proteins or a live recombinant vector, with the genetic material from one or more of the main antigens of Brucella sp. Inserted in its genome are the herpes or recombinant smallpox viruses. The British application GB2227936 protects a number of forms of administration of some of the Brucella sp. proteins, but none of these is SOD.

United States invention U.S. Pat. No. 5,824,310 discloses the use of LPS of B. abortus as a coadjutant. This invention patent application does not include the use of the SOD protein.

The invention patent application with United States priority at the world patent office WO03104468, discloses a vector system based on the Semliki Forest virus (SFV), in addition to its use in an expression system directed at the central nervous system (CNS) and the related pharmaceutical formulation for drug release in the CNS. The is invention demonstrated the potential use of alpha virus vectors as vectors for the CNS. A vector that penetrates the CNS and expresses a cloned gene that acts on the CNS, providing non-invasive effective treatment, is protected. The United States application uses neither the SOD protein nor the gene. It does, however, use the viral system as a vector, but this system was already protected in Liljestrom's invention patent application (U.S. Pat. No. 5,739,036).

The invention patent application at the world patent office WO9909192 discloses and protects a method for transforming a selected cell with a particular nucleic acid. For this purpose, particles of the Semliki Forest virus were used to infect in vivo in a selective way. The target cells are smooth cardiac muscle cells and cardiomyocytes after an angioplasty. The aim is that the nucleic acid encodes for a restenosis inhibitor, the thymidine kinase of the herpes simplex virus. This patent application does not work with the SOD protein.

United States invention U.S. Pat. No. 6,566,093 discloses a new expression vector to be used as a vaccine. It is of DNA type and is based on part of the genome of an alphavirus. This patent protects the use and introduction methodology of an exogenous gene in the said expression system. This technology does not interfere with our proposal, since the DNA construct is different from that synthesized in the present invention.

The invention patent application at the world patent office WO95/27069 protects an injectable pharmaceutical composition that comprises an RNA type alpha virus molecule. It contains a sequence of exogenous RNA that encodes for an antigen of the herpes simplex and influenza virus. In addition, a naked RNA type vaccine composition is protected, formulated with lipids that can be absorbed by inert particles together with the sequence of the exogenous antigen, where the Herpes antigen is HSVgD and that of the influenza is hemagglutinin. The present invention wishes to protect a different pharmaceutical formulation.

### DISCLOSURE OF THE INVENTION

Two vaccines have been developed. The first is based on the genome of the Semliki Forest virus against the intracellular bacterium Brucella abortus, using as an antigen a specific protein of this bacterium called Cu/Zn superoxide dismutase, which is capable of inducing a protective immune response against the pathogenic strain B. abortus. The is second vaccine only incorporates the RNA of the virus with the sequence of the protein.

To create this expression system, it is necessary to subclone the DNA segment that encodes the Cu/Zn rSOD protein (sodC gene) in the plasmid that carries the sequence of the viral replicase (pSFV4.2). Subsequently, the 3 plasmids that encode the recombinant Semliki Forest virus are transcribed in vitro. After the transcription in vitro, the SOD gene expression analysis is conducted on the basis of the RNA replicon (pSFV4.2-SOD). The results obtained indicate that the SOD protein is expressed with similar effectiveness by the cells of an animal immunized with this RNA. Then the viral particle (rSFV4.2-SOD) is packaged using the 3 transcribed RNA within one cell line (COS-7), from which the chimeric viral particles of the culture medium are purified.

Trial results indicate vaccine effectiveness. These expression systems provide protective immunity and are capable of inducing a response that is greater than those obtained with conventional vaccines, thus solving the continuing problem of the biosecurity of high-efficiency molecular systems.

This invention of a vaccine against the bacterium Brucella abortus, as one of the products of this process, includes a number of stages, as follows.

A. Obtaining the Antigens

The strain RB51 was used to extract the total proteins from Brucella abortus, and particularly to obtain the Cu/Zn superoxide dismutase (SOD) protein. The procedure considers the culture of the strain for a period of 24 hours and its subsequent harvest. The pellet is treated with methanol and a hypertonic solution to stop bacterial activity; then it is sonicated and centrifuged in cold conditions, the supernatant containing the already lysed bacteria. This pellet is treated with phenylmethylsulphonyl fluoride, a protease inhibitor (PMSF) and dialyzed, in order to obtain the proteins. Finally, the proteins are concentrated with polyethylene glycol in dialysis bags with a retention capacity of molecular weights over 3500. This protein solution contains the Cu/Zn SOD protein used as a control.

B. Expression of the Recombinant Cu/Zn Superoxide Dismutase Protein

To obtain the recombinant Cu/Zn SOD protein of Brucella abortus, a gene bank was generated with the strain B. abortus 2308. Then with a 20-base probe a sequence was cloned of 1.4 to 1.6 kb, containing the gene with its promoting sequence. This gene is expressed in E. coli DH5 bacteria, transformed by electroporation with the plasmid pBSSOD, which contains the gene that encodes the Cu/Zn SOD protein (sodC).

To obtain the protein, the bacterium must be cultured, then collected from the culture medium and the supernatant is added to an anionic exchange column that does not possess affinity for the Cu/Zn SOD protein. The supernatant elutes the Cu/Zn SOD protein and is treated with polymixine B so as to eliminate the bacterial lipopolysacharide. Then this solution is dialyzed against PBS buffer, in order to finally analyze the purity of the protein obtained using an SDS-PAGE gel and the concentration is determined through the Bradford method.

C. Stages for the Preparation of the Plasmids and their Expression

This stage is carried out in two parts: first there is the creation of an expression vector that encodes for the SOD protein from the plasmid that contains the viral replicase genes of the Semliki Forest virus. A second stage implies a second expression system, also based on plasmids from the same virus; these carry other genes necessary for viral replication.

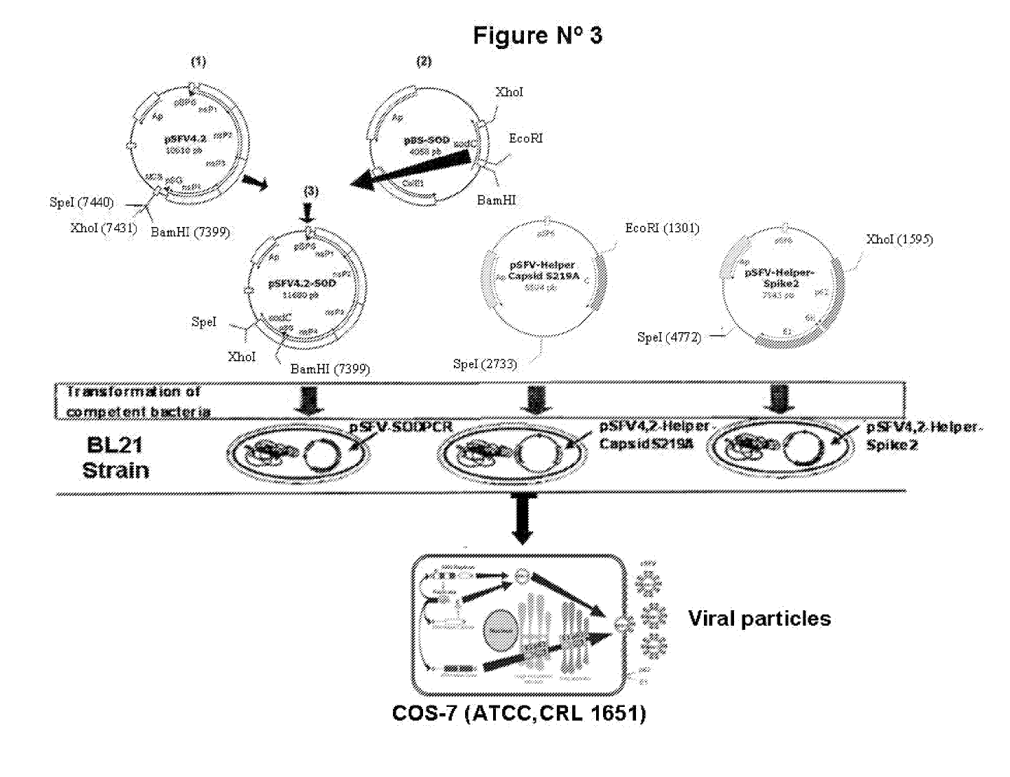

In order to generate the expression system, the competent bacteria must be prepared. The strain used is E. coli BL21 for the two plasmids from the first stage, the transformation protocol implying the use of CaCl₂. The construction of the plasmid pSFV4.2-SOD is carried out using the gene that encodes the Cu/Zn superoxide dismutase protein of B. abortus (sodC) that is obtained from the plasmid pBSSOD, previously developed in the invention, and from the plasmid pSFV4.2. Once the plasmid has been constructed, the already competent bacteria are transformed using classic methods that are widely known in the field. FIG. 3 shows a general scheme of the process up to the point where the suicidal viral particles are obtained: (1) The plasmid is constructed using the plasmid pSFV4.2. (2) The plasmid pBSSOD is digested with the same restriction enzymes and is synthesized after the gel extraction of the insert between 1000 and 1200 pb (sodC) whose gene encodes the Cu/Zn Superoxide Dismutase protein of B. abortus. In (3), the ligation of the insert takes place in a range of between 1000 and 1200 pb in the plasmid pSFV4.2. In (4) the purification of each plasmid, in Vitro transcription and transfection are carried out.

The second expression stage is the construction of the two viral structural plasmids, for which the vectors pSFV-Helper Spike2 (7543 pb) and plasmid pSFV-Helper Capsid S219A (5504 pb) are used.

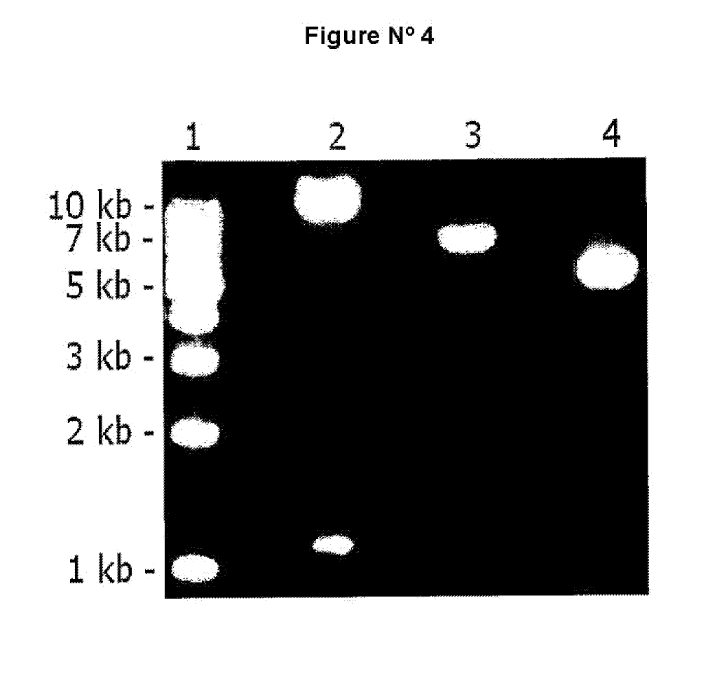

In order to analyze the plasmidial constructs used to create the expression system, is based on the Semliki Forest virus, these constructs are digested with restriction enzymes and subsequently examined using electrophoresis in agarose gel at 1%. FIG. 4 shows that the linealized plasmids pSFV-Helper Spike2 and pSFV-Helper Capsid S219A concur with the respective theoretical molecular weight values (Lines 3 and 4); in addition, Line 2 confirms the presence of the insert in a range between 1000 and 1200 pb in the plasmid pSFV4.2-SOD (11680 pb), which is digested with two restriction enzymes simultaneously. In this same figure, the agarose gel (1%) analysis of the constructs is individualized after digestion with endonucleases:

Line 1: 1 kb DNA molecular weight standard,
Line 2: Plasmid pSFV4.2-SOD digested with XhoI and BamHI,
Line 3: Plasmid pSFV-Helper Spike2 digested with XhoI,
Line 4: Plasmid pSFV-Helper Capsid S219A digested with EcoRI.

D. In Vitro Transcription

Before the plasmids can be transcribed, they must be linealized, for which the restriction enzyme (SpeI) was used in this invention. The in vitro transcription was carried out using a commercial kit. Transfection to the cell line COS-7 (ATCC, CRL 1651) was done by means of cationic liposomes.

E. Expression Analysis of the RNA Transcribed from the Plasmid pSFV4.2-SOD

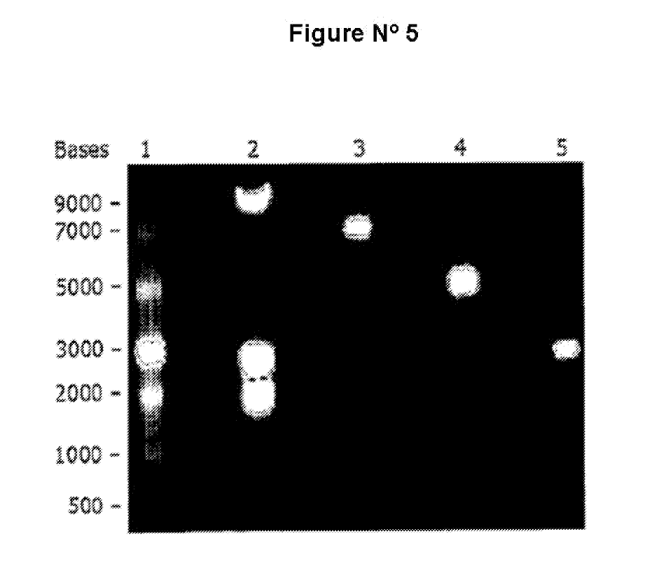

The RNA transcribed from the plasmid pSFV4.2-SOD, as well as the RNA from the plasmids pSFV-Helper-Spike2 and pSFV-Helper-Capsid S219, are obtained by in vitro transcription, as previously described. This procedure is specifically developed in the application example. FIG. 5 reveals the effectiveness of the in vitro transcription. The sizes of the RNA transcribed from the plasmids pSFV-Helper Spike2, pSFV-Helper Capsid S219 and pSFV4.2-SOD, are as expected.

FIG. 5 presents in detail the analysis of the RNA transcribed from the plasmids under study. The 1% agarose gel is subjected to electrophoresis for 30 minutes at 40 mA. Both the standard RNA and the transcribed RNA must be previously incubated with a loading buffer and heated to 65° C. for 3 minutes before being spread in the gel. Specifically, FIG. 5 shows the following:

Line 1: RNA molecular weight standard,
Line 2: Positive control of transcription,
Line 3: RNA transcribed from the plasmid pSFV4.2-SOD,
Line 4: RNA transcribed from the plasmid pSFV-Helper Spike2,
Line 5: RNA transcribed from the plasmid pSFV-Helper Capsid S219A.

Line 2 shows the correct transcription in vitro of the positive control, and in addition the correct transcription of each plasmid, the latter possessing the desired sizes.

F. Western Blot

In order to visualize the expression of the recombinant SOD protein, a Western Blot is carried out. For this purpose, electrophoresis of the proteins must first be conducted in a polyacrylamide gel. Once the proteins are transferred to the nitrocellulose paper, the non-specific sites are blocked, using non-fat milk dissolved in a saline phosphate tampon plus Tween 20. Then the nitrocellulose paper must be incubated under agitation for a period of time with a monoclonal antibody against SOD. It must then be incubated with a second mouse anti rabbit IgG antibody, marked with peroxidase. Finally, the incubation-transferred paper is revealed in a solution of Diaminobenzidine (DAB) in a PBS buffer, in which a positive reaction at 18 kD should be observed.

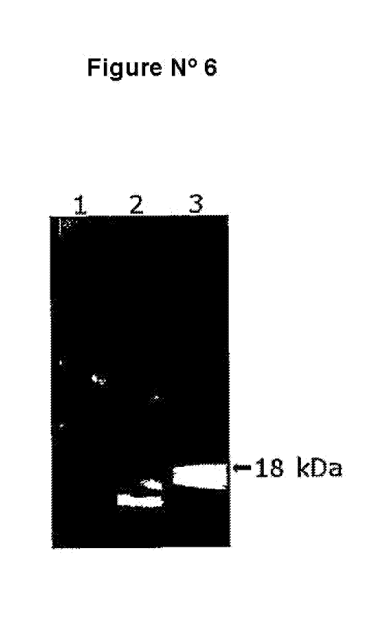

FIG. 6 shows the Western Blot for the expression analysis of the Cu/Zn rSOD protein from the RNA replicon. A monoclonal antibody against the Cu/Zn rSOD protein is used for the Western Blot analysis and the pure Cu/Zn rSOD protein is used as a positive control.

Line 1: Negative control (cells without transfection with transcribed RNA),

Line 2: Sample (cells transfected with transcribed RNA pSFV4.2-SOD),

Line 3: Positive control (Cu/Zn rSOD protein).

G. Expression Analysis of the RNA Replicon

In order to demonstrate that the RNA transcribed from the plasmid pSFV4.2-SOD has the capacity to express the recombinant protein Cu/Zn superoxide dismutase (rSOD), it is transfected with RNA from the plasmid pSFV4.2-SOD to the cell line J774 (ATCC, TIB-67). Once transcribed, the expression of the Cu/Zn rSOD protein is detected within this cell line using a Western Blot.

Line 2 of FIG. 6 demonstrates positively the presence of the Cu/Zn rSOD protein within the cell line J774.

H. Production of Suicidal Viral Particles from the Semliki Forest virus

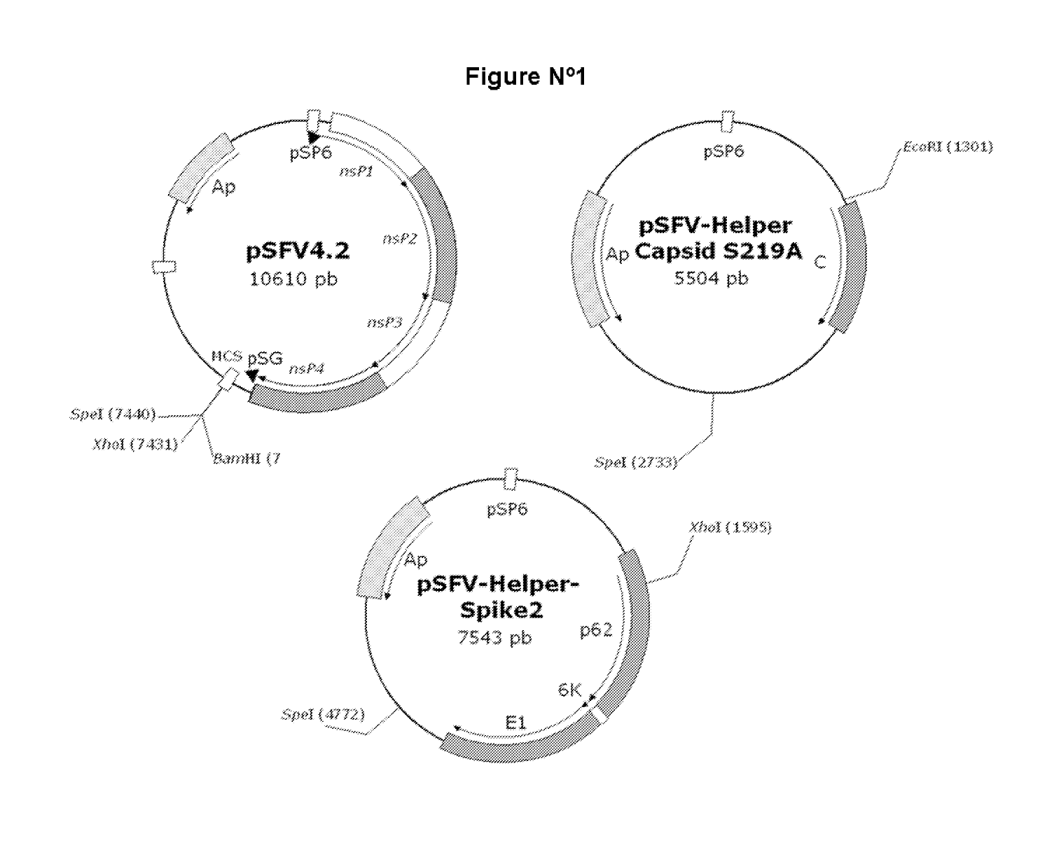

The Semliki Forest virus is modified genetically in order to produce a suicidal viral particle, which can be used as a vector for the expression of heterologous proteins in animals. The genetically modified virus is found encoded in three plasmids: pSFV4.2, pSFV-Helper-Capsid and pSFV-Helper-Spike. FIG. 1 presents the expression vectors based on the Semliki Forest virus.

The plasmid pSFV4.2 contains four genes that encode the Semliki Forest virus replicase (nsP1-4); this plasmid lacks the structural genes of the virus (C, p62, 6K and E1). The plasmids pSFV-Helper-Spike2 and pSFV-Helper-Capsid S219A lack the genes that encode the viral replicase, but possess the structural genes of the virus. The three plasmids have the following characteristics in common:
SP6 promoter to be transcribed in vitro,
A cut site of the restriction enzyme SpeI to linealize the plasmids before transcription,
An ampicillin resistance gene (Ap).

Each plasmid possesses an SP6 promoter that allows it to be transcribed in vitro, so that RNA molecules are obtained from each one. The plasmid pSFV4.2 possesses a multiclonage site, into which a gene can be inserted that encodes the SOD protein. The RNA of the plasmid pSFV4.2 corresponds to the replicon vector, a subgenomic promoter followed by the heterologous genes of interest (SOD) and the 5′ and 3′ ends required for the replication of the genome, available in the three RNAs. The RNA of the plasmid pSFV-Helper-Capsid contains a subgenomic promoter, followed by the genes that codify for the proteins of the virus capsid. The RNA of the plasmid pSFV-Helper-Spike also possesses a subgenomic promoter, followed by the genes of the transmembrane proteins of the virus envelope.

The three transcribed RNAs are cotransfected to the eucariot cell line COS-7 and subsequently translated, and they then begin the packaging of the viral particles with the information from the protein of interest. Because of a genetic modification, these viruses possess a limited genome that only consists of the RNA of the replicon vector, since only this has the sequence of the encapsidation signal. In this way, the virus is prevented from developing a productive infection, providing the system with high biosecurity. In addition, a mutation has been introduced into the gene that codifies for the protein p62 (Arg₆₆→Leu) that impedes the cleavage of this protein by the host proteases. Thus the viruses obtained are conditionally infective. Therefore the cotransfection of a cell with the three RNAs (Replicon, Helper-Spike, Helper-Capsid) induces the packaging and release by gemation of the recombinant Semliki Forest virus, which only encapsidates the replicon RNA, given that only this possesses the encapsidation signal.

In the present invention an expression system and two vaccines have been developed by constructing a new plasmid, called pSFV4.2-SOD from the gene that codifies for the protein Cu/Zn Superoxide Dismutase and from the plasmid pSFV-4.2. To this purpose, the plasmids pSFV4.2-SOD, pSFV-Helper-Capsid S219A and pSFV-Helper-Spike2 were purified and transcribed in vitro. The recombinant Semliki Forest virus was obtained from the cells cotransfected with the RNA transcribed from the plasmids pSFV4.2-SOD, pSFV-Helper-Capsid S219A and pSFV-Helper-Spike2.

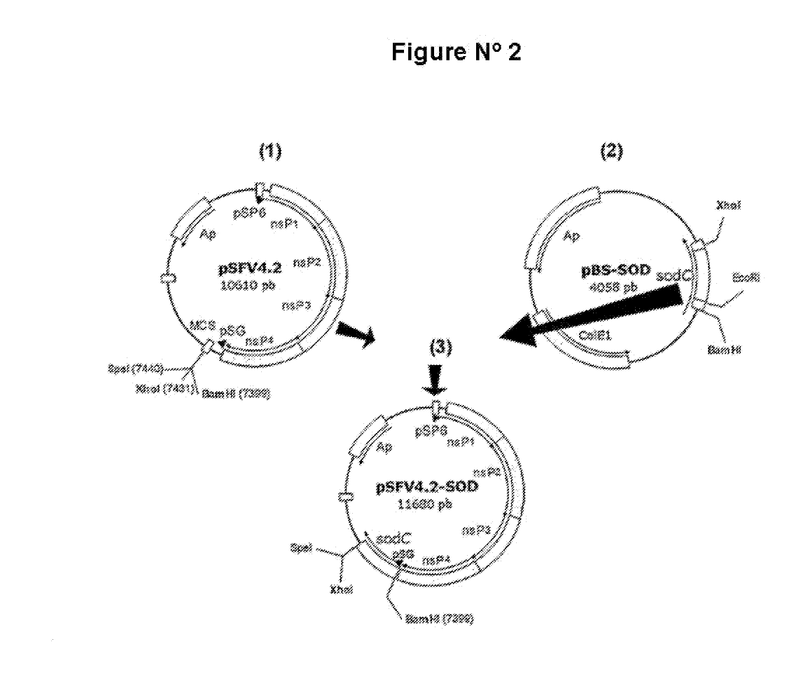

FIG. 2 shows the construction of the plasmid pSFV4.2-SOD. To construct this plasmid, the plasmid pSFV4.2 was used, previously digested with the restriction enzymes BamHI and XhoI (1), before its ligation with the fragment obtained from the digestion of the plasmid pBSSOD with the same restriction enzymes (2), which was synthesized after the extraction of the insert gel between 1000 and 1200 pb (sodC). The fragment of 1.1 kb contains the sodC gene that codifies the Cu/Zn superoxide dismutase protein of B. abortus. In (3), the ligation of the insert takes place in the range included between 1000 and 1200 pb in the plasmid pSFV4.2, previously digested with the same restriction enzymes.

To demonstrate the viability of the vaccine, female mice from strain BALB/c were immunized. The naked RNA replicon (pSFV4.2-SOD) was administered intramuscularly and the replicon RNA wrapped in the Semliki Forest virus (rSFV4.2-SOD) was administered intraperitoneally.

The expression study of the SOD protein, through the replicon RNA packaged in the Semliki Forest virus in vitro, used the cell line COS-7 (ATCC, CRL 1651), which are African green monkey kidney fibroblasts, and the cell line J774 (ATCC, TIB-67), which are mouse macrophages. Both cell lines were cultivated in a complete DMEM medium. 3 bacterial strains were used for this invention:
the bacterium Brucella abortus 2308, which is virulent,
the bacterium Brucella abortus RB51, an attenuated strain of Brucella abortus 2308, and
E. Coli BL21, that over expresses the SOD protein in a recombinant form.

The plasmids used were pSFV4.2, pSFV4.2-Helper-Spike2 and pSFV4.2-Helper-Capsid S219A (see FIG. 1).

I. Production of Recombinant Semliki Forest Virus.

The packaging of the Semliki Forest virus is done inside the cell line COS-7, for which purpose it is necessary to cotransfect with the RNAs transcribed from the plasmids pSFV4.2-SOD, pSFV-Helper-Capsid S219 and pSFV-Helper-Spike2. The transfection is carried out through cationic liposomes. Subsequently the transfection mixture is stirred and the cotransfected cells are then incubated in RPMI medium. The viral particle formed inside the COS-7 cell is released into the culture medium, from which it is purified using a discontinued sucrose gradient. Finally, it is necessary to dilute the fraction in which the viral particles are found.

The visualization and identification of the viral particles of recombinant Semliki Forest virus is carried out using an electronic microscope. The same cell line without transfection is used as a negative control. What is obtained from the sucrose gradient of this control is observed in the electron microscope.

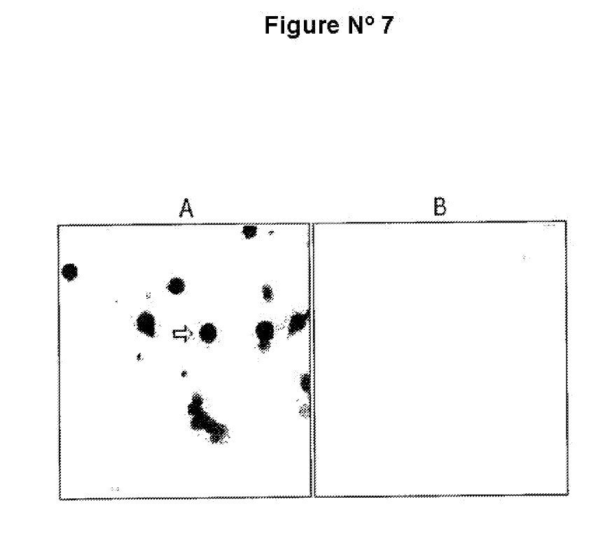

FIG. 7 shows the electron microscopy image of a sample that contains viral particles of recombinant Semliki Forest virus. FIG. 7A is the photograph of a sample that contains viral particles purified in a sucrose gradient, and FIG. 7B shows the negative control of the previous sample. These samples must be previously stained with a phosphotungstic acid solution, which is the differential stain for the virus.

FIG. 7A shows the presence of particles with a rounded form and greater density than the rest of the sample, whose size is similar to that of the Semliki Forest virus particles, which are not observed in FIG. 7B, corresponding to the control.

J. Immunization Experimental Scheme

To determine the effectiveness of the vaccines, a trial was carried out with female mice of the BALB/c strain, which were immunized with viral particles of the Semliki Forest virus or with the naked RNAs that encode for the rSOD protein.

Table 1 specifies the trial groups for the constructs designed from the Semliki Forest virus. The first group (I) of individuals in the trial were immunized with the sequences of naked RNAs, that is, group I.A. The immunization sequence encodes for the rSOD protein from the RNA rSFV4.2-SOD. A second group, called I.B, was subjected to a trial with naked RNA, but with the construct that does not encode for the SOD protein (rSFV4.2). The second group (II) in the study was immunized with viral particles, specifically the individuals II.A, that were subjected to viral immunization, the genetic material of which carried the genes that encode for the rSOD proteins and was constructed from the plasmid pSFV4.2-SOD. Group II.B was also subjected to viral action, but in this case the genome only carried the genes that encode for the protein complex of the viral replicase. In addition, phosphate buffered saline (PBS) was used as a negative control, at a pH of 7.4. The viral particles must be previously activated using a solution of succinic acid at pH 4.5.

TABLE I
Experimental groups and immunization method

| | | | Vaccine active | Immunization |
|---|---|---|---|---|
| Group | Trial | Vector | component | method |
| I | I.A | Naked RNA + rSOD | pSFV4.2-SOD | Intramuscular |
| | I.B | Naked RNA | pSFV4.2 | Intramuscular |
| II | II.A | Viral particles of the | pSFV4.2-SOD | Intraperitoneal |
| | | SF virus + rSOD | | |
| | II.B | Viral particles of the | pSFV4.2 | Intraperitoneal |
| | | SF virus | | |
| Control | | Phosphate buffered | PBS | Both methods |
| | | saline | | |

The cellular immune response of the mice immunized with the expression systems is evaluated, for which purpose the proliferation of spleen lymphocytes in the mice is measured in reaction to antigens such as the Cu/Zn rSOD protein and total proteins of Brucella abortus RB51. The way in which these two antigens are obtained has been described in sections A and B of the description of the invention in the descriptive memory, entitled “Obtaining the antigens” and “Expression of the recombinant Cu/Zn superoxide dismutase protein”.

The proliferation is determined by measuring the incorporation of thymidine [³H] into the DNA of the mouse spleen cells. The cells are induced to divide actively in the presence of the antigen. Cell suspension must be spread in microplates and the antigen, corresponding to the Cu/Zn rSOD protein or total proteins of Brucella abortus is strain RB51. Splenocytes are cultured as a positive control, and as a negative control only the complete culture medium is incubated. The cells are cultured and then the lymphocytes are harvested to include them in the scintillation solution. Finally, the stimulation index (SI) is determined through the obtention of the quotient between the value obtained in counts per minute (cpm) of the experimental group with the cpm obtained in the negative control of the same experimental group.

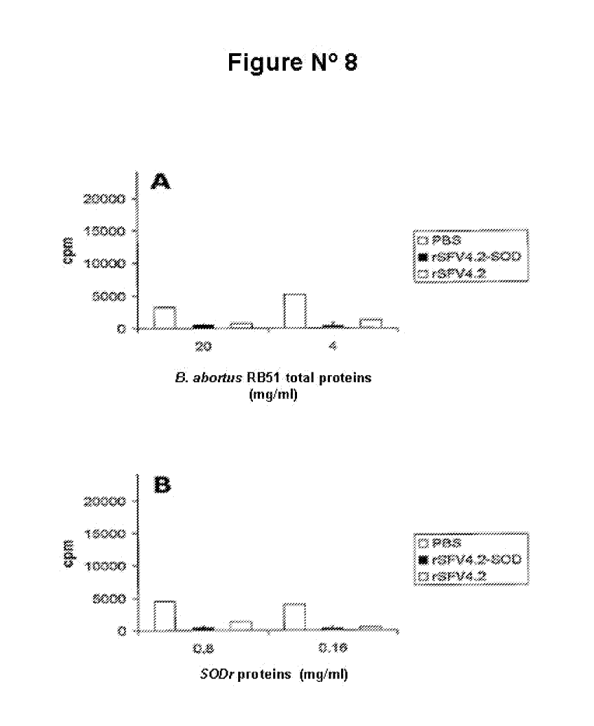

FIG. 8 shows graphically the results of the proliferation of the spleen lymphocytes in mice immunized with a naked RNA vaccine from the sequences that encode for the SOD protein, viral replicase and the buffer (rSFV4.2-SOD, rSFV4.2 and PBS). The lymphoproliferation study is carried out for 28 days after the second immunization, the lymphocytes being cultured in the presence of the total protein of Brucella abortus RB51 (FIG. 8, Graphic A) and the protein Cu/Zn rSOD (FIG. 8, Graphic B). In Graphic A, spleen lymphocyte proliferation is not observed in the mice immunized with rSFV4.2-SOD, nor is it observed in the controls rSFV4.2 and PBS. In Graphic B of the figure it can be seen that the lymphocytes of mice immunized with the recombinant protein rSFV4.2-SOD, as in the previous case, did not proliferate significantly in response to the antigen.

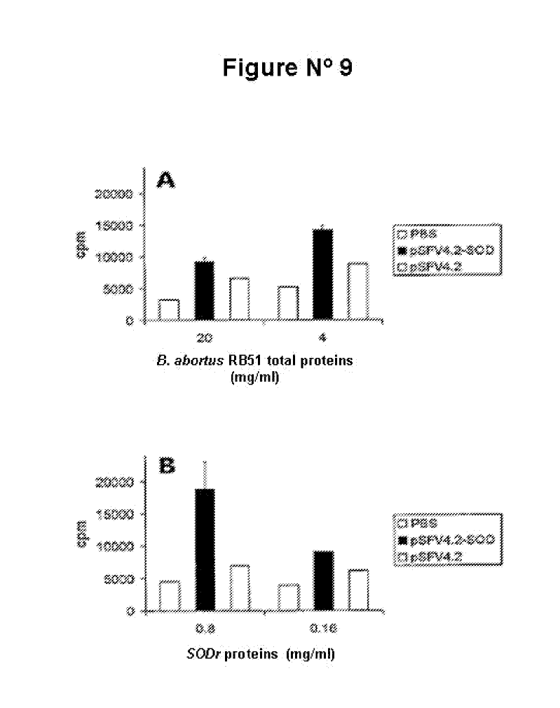

FIG. 9 shows graphically the results of the spleen lymphocyte proliferation in mice immunized with the vaccine that contains the genetically modified virus (pSFV4.2-SOD, pSFV4.2 and PBS). The lymphoproliferation was carried out 18 days after immunization, the lymphocytes being cultivated in the presence of the total protein of Brucella abortus RB51 (Graphic A) and the protein Cu/Zn rSOD (Graphic B). In Graphic A of the figure it is observed that the lymphocytes of the mice immunized with pSFV4.2-SOD proliferated more than the lymphocytes of the mice immunized with the controls pSFV4.2 and PBS. The maximum (14229 cpm) was obtained with a concentration of 4 μg/ml of the antigen, the value of which is significantly higher than that of the lymphocytes in the mouse control group immunized with pSFV4.2 (8794 cpm) and PBS (5254 cpm). In Graphic B of the figure it can be observed that there is a higher proliferative response from lymphocytes in mice immunized with pSFV4.2-SOD. The maximum (18876 cpm) was obtained with a concentration of 0.8 μg/ml of the antigen, the value of which was significantly higher than that of the lymphocytes in the mouse control group immunized with pSFV4.2 (7056 cpm) and PBS (4541 cpm).

### Protection Trial

The mice must be challenged with 10⁴ colony-forming units (CFU) of the pathogenic strain Brucella abortus 2308, injected intraperitoneally. The challenges were carried out 24 days after the second immunization in the case of the mice immunized with RNA replicon or pSFV4.2-SOD (Group I), in addition to their respective controls, and 36 days after immunization in the case of mice immunized with the recombinant Semliki Forest virus (rSFV-SOD) plus their respective controls (Group II). The protection trial was carried out 2 weeks later, for which the spleens of the mice in the trial were removed. Protection is expressed as the logarithm of the number of CFUs present in the dilution spread in the dish, in which it was possible to observe a maximum number of isolated colonies.

Table II shows the high efficacy of the expression systems to confer protection against the challenge from the pathogenic strain. The greatest protection efficacy against Brucella abortus occurred in mice immunized with rSFV4.2-SOD, where the presence of bacteria in the spleen was not observed. In addition, in the case of mice immunized with pSFV4.2-SOD, it was determined that a high level of protection was achieved.

TABLE II
Protection systems against the challenge
of strain 2308 of B. abortus

| | Log10 CFU of B. | |
|---|---|---|
| | Abortus 2308 in the | Log₁₀ Protection |
| Vaccine | spleen (average) | Units ^{b} |
| pSFV4.2-SOD | 2.23 ± 1.48 | 1.76 |
| pSFV4.2 | 4.62 ± 0.01 | — |
| rSFV4.2-SOD | 0 | 3.99 |
| PBS | 3.99 ± 0 | 0 |

This invention includes the development of two easy to use, highly efficient vaccines with a high level of biosecurity, whose response is greater than vaccines currently available on the market, such as the classic vaccines from attenuated organisms like strain RB51. The technology proposed is an alternative option for the development of one or more molecular vaccines against this bacterium.

Two options can be visualized in this invention: the first corresponds to the use of expression systems based on the Semliki Forest virus (SFV), which have extensively shown themselves to be excellent expression vectors of heterologous proteins inside eucariot cells. The second option is the use of naked RNA, a carrier of the information required for the synthesis of a heterologous protein, with the capacity to generate an immune response against Brucella abortus.

In the art, initiatives exist to massify the use of this type of technology in the pharmaceutical industry. However, no initiatives have been disclosed to eradicate B. Abortus, using RNA vaccines and even less using recombinant RNA viruses. There is therefore a permanent need to develop new formulations offering high biosecurity. This invention discloses two expression systems capable of inducing protective immunity greater than that generated by traditional vaccines, and, in addition, these systems are low cost as well as highly efficient, with a high level of biosecurity.

The invented expression system presents some additional advantages that establish the difference in the type of response induced in the immunized animal. This surprising expression system consists of a viral replicase encoded in the RNA replicon, which has the particularity that it synthesizes various copies of the genomic RNA, increasing even more the probability of the translation of the RNA molecule of interest. In addition, the viral particles based on the Semliki Forest virus have a high affinity with a broad spectrum of cell receptors, which allows them to enter a great diversity of cells. Some of these cells are crucial for the development of the protective immune response, as are antigen presenter dendritic cells, although the latter do not phagocytize as efficiently as the macrophages, increasing even more the efficiency of the immune system response.

This invention includes an expression system with a high level of biosecurity, because the virus is not self-replicating and its genome is constituted by an RNA replicon sequence that is not incorporated into the host genome, as its metabolism does not require DNA as an intermediary.

### APPLICATION EXAMPLES

### Example 1

### Extraction of Total Proteins from the Strain RB51 of Brucella abortus

The procedure for the extraction of bacteria included their culture. Once harvested, they were washed three times with sterile PBS at a pH of 7.2, centrifuged at 10000 rpm for 10 minutes at 4° C., eliminating the supernatant. The bacteria were inactivated in methanol at 60% for 24 hours. Subsequently the cells were washed again and kept for 24 hours at 4° C. in a hypertonic saline solution that contained NaCl (1 M), 0.1 sodium citrate and EDTA (0.5 mM). Then the cells were sonicated for approximately 15 minutes at 60 watts, and centrifuged at 10000 rpm for 10 minutes at 4° C. 0.2 mM of phenylmethylsulfonyl fluoride (PMSF) were added to the supernatant and the proteins were concentrated with polyethylenglycol in dialysis bags with a molecular weight retention capacity above 3500 kD. Then the fraction concentrated in this way was dialyzed against distilled water for two days, at the end of which it was centrifuged at 7500 rpm for 30 minutes at 4° C. Subsequently the proteins were quantified using the Bradford method and stored at −20° C.

### Example 2

### Expression of the Recombinant Cu/Zn Superoxide Dismutase Protein (see FIG. 6)

The Cu/Zn SOD protein of Brucella abortus was expressed in E. coli DH5 bacteria that were transformed by electroporation with the plasmid pBSSOD that contains the gene that encodes the Cu/Zn SOD protein (sodC). To obtain the protein the bacterium was cultured in LB broth plus 100 μg/ml of ampicillin for 12 hours at 37° C., with agitation. Subsequently the bacteria were collected from the culture broth and centrifuged at 3000 rpm for 20 minutes. The bacteria were re-suspended in 10 mM phosphate buffer at pH 7.6 plus 0.1% of Triton X-100 and were incubated at 37° C. for 12 hours, with agitation. The mixture was centrifuged at 10000 rpm for 20 minutes, and the recovered supernatant was then added to a column of anionic exchange, which has no affinity for the Cu/Zn SOD protein, most of the other proteins present in the supernatant being retained. The eluate obtained from the column was treated with polymixine B in order to eliminate the bacterial lipopolysaccharide. Finally, this solution was dialyzed is against PBS buffer, in order to analyze the purity of the protein obtained through an SDS-PAGE gel at 12% and its concentration through the Bradford method. The Cu/Zn rSOD protein was stored at −20° C.

### Example 3

### Construction of the Plasmid

Once the original gene of the Cu—Zn superoxide dismutase protein (SodC) had been obtained using restriction enzymes from the plasmid pUC19, the plasmid pSFV4.2-SOD was constructed. For this purpose, the plasmid PUC19 was subjected to digestion with the enzymes BamHI and XhoI for 2 hours at 37° C. From the digestion a fragment of 1.1 kb was obtained that contained the gene of interest, which was extracted from 1% agarose gel using a commercial kit. (see FIG. 4, Line 2). In addition, the plasmid pSFV4.2 was digested with the same restriction enzymes used previously and in the same conditions. At the end of incubation, the restriction enzymes were inactivated at 60° C. for 15 minutes. Subsequently, ligation was carried out, mixing in a proportion of 3:1 the 1.1 kb insert with the plasmid pSFV4.2, which presented a marker for the ampicillin antibiotic. This was previously digested using the ligase enzyme DNA T4 in ligase buffer DNA T4 10× plus 5 mM of ATP. The ligation mixture was incubated for 12 hours at 16° C. in darkness and used to transform competent E. coli BL21 bacteria. Ligation effectiveness was determined by culturing in dishes with LB medium that contained 100 μg/mL of ampicillin. Some of the colonies that grew were selected and cultured with agitation for 12 hours in LB broth with 100 μg/ml of ampicillin at 37° C. Then the plasmidial DNA was extracted using a commercial kit. The plasmid obtained was digested with the enzymes BamHI and XhoI, then analyzed using 1% agarose gel, which was observed under ultra violet light to confirm the presence of the 1.1 kb fragment.

### Example 4

### Transformation of Competent Bacteria

Competent E. coli BL21 bacteria were transformed by mixing 100 μl of the said bacteria with approximately 1 μg of plasmid, keeping them in ice for 30 minutes. They were subsequently incubated at 42° C. for 90 seconds, 400 μl of LB broth were added and once again they were incubated, this time for 45 minutes at 37° C. with agitation at 200 rpm. Finally the mixture was spread in a culture dish that contained LB agar plus 60 μg/ml of ampicillin and the bacteria were incubated for a period of 12 hours at 37° C.

### Example 5

### Competence Test

The bacterial strain E. coli BL21 was spread in Laurya Bertoni (LB) agar and was incubated at 37° C. for 16 hours. Then an isolated colony was selected from the dish and this colony was inoculated into a test tube with 5 ml of LB broth and then incubated for 12 hours at 37° C. with agitation at 220 rpm. Subsequently 1 ml of the medium was inoculated into a flask with 100 ml of LB broth and was incubated at 37° C. with agitation at 220 rpm until the broth acquired an optical density of 0.38 to 590 nm. Once the optical density was reached, the culture medium was centrifuged to 2500 rpm for 10 minutes and the supernatant was discarded. The bacteria were resuspended in 20 ml of CaCl₂ 0.1M at 4° C. They were incubated for 10 minutes in ice and centrifuged at 2500 rpm for this same period of time. The supernatant was discarded and the bacteria were resuspended in 4 ml of CaCl₂ (0.1 M) at 4° C. To preserve the bacteria, 850 μl of the previous suspension were mixed with 150 μl of sterile glycerol in a microfuge tube with a capacity of 1.5 ml, and then each tube was introduced into a container with liquid nitrogen. Finally, the frozen competent bacteria were stored at −80° C.

### Example 6

### Linearization of the Plasmid and In Vitro Transcription System

The linearization of the plasmids was carried out by digestion with the enzyme SpeI at 37° C. for one hour. The linearized plasmids were purified from the cut mixture, then a mixture volume was added containing 25% phenol, 24% chloroform and 1% isoamilic alcohol, and the mixture was agitated energetically. It was subsequently centrifuged at 4650 rpm and the water phase was recovered, from which the plasmid was extracted by precipitation using 2.5 volumes of ethanol at 70% plus 0.05 volumes of sodium acetate 3M. The plasmid was resuspended in deionized water treated with 0.2% of diethilpyrocarbonate (DEPC). Then the in vitro transcription was carried out using a commercial kit.

The mixture of 5 μg of linearized plasmid was treated with 10 μl of transcription buffer SP6 5×; 5 μl of dithiotreitol (DTT) 100 mM; 50 units of recombinant ribonuclease inhibitor; 2.5 μl of rATP 10 mM, rCTP 10 mM and rUTP 10 mM plus 2.5 μl of rGTP 1 mM; 5 μl of Cap analogue 5 mM, Ribo m⁷G; 40 units of RNA polymerase SP6 and was brought to a final volume of 50 μl with nuclease-free water. This mixture was then incubated for 2 hours at 37° C. When the transcription reaction was completed, the transcribed RNAm was purified by precipitation with 0.72 volumes of isopropanol at −20° C. plus 0.2 volumes of sodium acetate 3M (pH 4.8), and incubation was again is carried out, this time for 10 minutes at room temperature. Subsequently, the transcribed RNA was centrifuged for 15 minutes at 4650 rpm, and then precipitated. For this stage, it was washed with ethanol at 75% and centrifuged at 4650 rpm for a period of 15 minutes. The precipitated RNA was resuspended in TE buffer at pH 7.5. The size of the transcribed RNA was verified through resolution by electrophoresis in 1%. agarose gel. The sample was previously incubated with a loading buffer for 3 minutes at 65° C. before being spread in the gel. The gel was analyzed in an ultraviolet transilluminator in which the size of the transcribed RNA was compared to that of the RNA molecular weight standard. The transcribed RNA was aliquoted and stored at −80° C. (see FIGS. 4 and 5).

### Example 7

### Cell Transfection

The transfection was carried out using cationic liposomes, for which cell lines COS-7 (ATCC CRL-1651) and J774 (ATCC TIB-67) were cultured in a complete DMEM medium, until approximately 4×10⁶ cells per ml were obtained. The cells were detached and transferred to dishes for cell cultures, where the cells were incubated in a culture medium until a confluence of 80% was reached. This culture medium was then replaced by the complete modified DMEM medium and was incubated for 5 to 10 minutes in a humid atmosphere at 37° C. with 5% CO₂. After incubation, the medium was replaced by a transfection mixture that contained 9 μg of lipofectamine plus 2-5 μg of transcribed RNA in complete modified DMEM medium. The transfected mixture was incubated for 2 hours.

### Example 8

### Expression of the RNA Transcribed from Plasmid pSFV4.2-SOD

The transcribed RNA was transfected in cell line J774 (ATCC, TIB-67), following the same transfection steps as with liposomes. The transfected cells were detached by mechanical means and lysed with a loading buffer, used in the electrophoresis of proteins in polyacrylamide gels. The previous mixture was heated to 100° C. for 5 minutes and then loaded in a polyacrylamide gel to electrophoretically separate the proteins from the sample. The expression of the Cu/Zn SOD protein in the transfected cell line was verified using a Western Blot (FIG. 5), which procedure is described in Example 10. As a first antibody, a mouse IgG monoclonal antibody was used against the Cu/Zn SOD protein (FIG. 7).

### Example 9

### Preparation of the Polyacrylamide Gel SDS-PAGE

The polyacrylamide gels, constructed on a gel support, consist of a separator gel and a concentrator gel. The former was prepared at 12% by mixing 2 ml of an acrylamide solution at 30%, plus 1.3 ml of Tris buffer at pH 8.8 and 0.05 ml of sodium dodecyl sulphate at 10%. Polymerization was initiated by adding 0.05 ml of ammonium persulphate and 0.002 ml of EDTA. The concentrator gel, prepared by mixing 0.17 ml of the acrylamide solution at 30% plus 0.13 ml of Tris-HCl at pH 6.8 and 0.01 ml of SDS at 10%, was added to the polymerized separator gel. Polymerization began by incorporating 0.01 ml of ammonium persulphate and 0.001 ml EDTA. When the gel polymerized completely, the sample, that had previously been mixed with loading buffer in a proportion of 1:10 and heated to 100° C. for 5 minutes, was loaded. A run buffer at room temperature and at 100 volts was used for the electrophoresis (see FIG. 5).

Polyacrylamide gel staining was carried out after electrophoresis. The gel was stained with a Coomassie blue solution at 0.5% plus 45% methanol and 10% acetic acid. The stain was then removed from the gel with a destaining solution that contained 10% methanol and 10% acetic acid dissolved in distilled H₂O.

### Example 10

### Western Blot (see FIG. 9)

In preparation for the Western Blot, protein electrophoresis was carried out on a polyacrylamide gel, described in Example 9. The gel was dismantled and disposed on a sheet of nitrocellulose paper of the same size. The gel was placed on a support for Western Blot in order to introduce it into an electrophoresis chamber that contained a transfer buffer. The operating conditions to carry out the transfer were one hour at 250 mA at room temperature. Once the proteins were transferred to the nitrocellulose paper, non-specific sites were blocked using non-fat milk at 5%, dissolved in phosphate buffered saline (PBS) plus 0.3% Tween 20, then they were incubated for 12 hours at 4° C. Subsequently, the nitrocellulose paper was incubated for 3 hours with the first monoclonal antibody against SOD in diluted form, which was in PBS buffer plus 0.03% Tween 20 and 5% non-fat milk at room temperature, with agitation.

The nitrocellulose paper was washed 3 times for five minutes with a PBS buffer and 0.03% Tween 20 under agitation. Then it was incubated for an hour with a second mouse anti rabbit IgG antibody marked with peroxidase, diluted in PBS buffer and 0.03% Tween 20, then washed again under agitation. Finally, the paper transferred by incubation was revealed in a solution that contained 10 mg/ml diaminobenzidine (DAB) and 0.3% hydrogen peroxide in PBS buffer.

### Example 11

### Production of Recombinant Semliki Forest Virus

The packaging of the Semliki Forest virus was carried out inside the cell line COS-7 (ATCC, CRL 1651), for which purpose it was cotransfected with the RNAs transcribed from plasmids pSFV4.2-SOD, pSFV-Helper-Capsid S219 and pSFV-Helper-Spike2. The transfection was done using liposomes, as described in Example 7. Then the transfection mixture was removed and the dish was washed with 2 ml of incomplete modified RPMI. Finally, the cotransfected cells were left incubating in complete modified RPMI for 24 hours in a humid atmosphere at 37° C. with 5% CO₂. The viral particle formed inside the COS-7 cell was released into the culture medium, from which it was purified using a discontinuous sucrose gradient. The gradient was prepared in an ultracentrifuge tube, to which was first added 1 ml sucrose at 55%, then 3 ml sucrose at 25%, and then 9 ml of the culture medium. The sucrose gradient was subjected to a centrifugation of 135000 rpm for 90 minutes in an ultracentrifuge. To recover the fraction that contained the viral particles, the culture medium was carefully removed from the gradient surface and then 0.8 ml sucrose at 55% was aspirated from the bottom of the tube. Then 1 ml was again aspirated from the bottom of the tube. This latter fraction, containing the viral particles, was then diluted to half in TNE buffer and stored in 50 μl aliquotes at a temperature of −80° C. Subsequently the viral particles were visualized in an electron transmission microscope, having been previously stained with a solution of phosphotungstic acid at 1%. At the same time, a negative control of this experiment was carried out, in which the same cell line was used, that is, the one not cotransfected with the transcribed RNAs. What was obtained from the sucrose gradient of this control was also observed under the electron transmission microscope (see FIG. 7).

1. A veterinary pharmaceutical formulation from an RNA viral vector system CHARACTERIZED because the vector system comprises the following constituents:
a. recombinant RNA particle as the active component, which encodes at least one Cu/Zn superoxide dismutase protein pathogenic bacteria from ruminants
b. at least one alphavirus RNA belonging to the family of Semliki Forest virus, and is carrier of the active component of this formulation, and/or
c. cationic liposomes as vehicle and
d. substances such as pharmaceutically acceptable excipients.

2. A veterinary pharmaceutical formulation from naked RNA CHARACTERIZED because the vector system comprises the following components:
a. recombinant RNA molecule as the active ingredient naked, carrying a sequence for the synthesis of at least a recombinant Cu/Zn superoxide dismutase of Brucella abortus and some genes of Semliki Forest virus,
b. optionally cationic liposomes as a vehicle for the formulation and
c. substances such as pharmaceutically acceptable excipients.

3. A veterinary pharmaceutical formulation from a vector viral RNA in accordance with claim 1, CHARACTERIZED because comprises a chimeric virus as a vector from Semliki Forest virus, which carries an RNA sequence exogenous and consists of the genes of the enzyme SOD Brucella abortus genes encoding the replicase protein complex viral genes encoding the capsid protein and gene encoding the spike protein of the capsid.

4. A veterinary pharmaceutical formulation from an RNA viral vector system in accordance with claim 1, CHARACTERIZED because the RNA sequence of the chimeric virus comprises RNA transcribed from plasmids pSFV4.2-SOD, pSFV-Helper-S219 and pSFV Capsid-Helper-Spike2.

5. A veterinary pharmaceutical formulation from an RNA viral vector system in accordance with claim 1, CHARACTERIZED because the RNA containing the nucleotide sequence encoding the protein Cu/Zn SOD, the chimeric alphavirus comprises a size between approximately 1.4 to 1.6 Kb.

6. A veterinary pharmaceutical formulation from an RNA viral vector system in accordance with claim 1, CHARACTERIZED because the RNA containing the nucleotide sequence encoding the protein Cu/Zn SOD, the chimeric alphavirus comprises a size between approximately 1.4 to 1.6 kb, which is isolated by the action of restriction endonucleases and XhoI and BamHI.

7. A veterinary pharmaceutical formulation from an RNA viral vector system in accordance with claim 1, CHARACTERIZED because it is useful for the treatment of bacterial diseases, specifically ruminants.

8. The pharmaceutical formulation containing part of Semliki Forest virus genome in accordance with claims 1 and 2, CHARACTERIZED because the route of administration is injection.

9. The pharmaceutical formulation containing part of Semliki Forest virus genome in accordance with claims 1 and 2, CHARACTERIZED because the information to synthesize the active ingredient of this formulation is contained in the plasmid pSFV4.2-SOD.

10. The veterinary pharmaceutical formulation from naked RNA in accordance with claim 2, CHARACTERIZED because the nucleic acid construct comprising the protein coding for superoxide dismutase of Brucella abortus.

11. A recombinant RNA molecule from Semliki Forest virus genome, CHARACTERIZED because the exogenous RNA sequence transcribed in vitro is able to express an antigenic polypeptide within a cell of a mammal, specifically, in a ruminant.

12. The recombinant molecule of RNA from Semliki Forest virus according to claim 11 CHARACTERIZED because the viral particles comprising part of its genome, the RNA transcribed from plasmid pSFV4.2-SOD.

13. A system of genetic material RNA vector according to claims 1 and 2 CHARACTERIZED because it comprises part of Semliki Forest virus genome and the gene that encodes Cu/Zn SOD of pathogenic bacteria.

14. A transformed mammalian cell line, CHARACTERIZED because the transformed cell line is COS-7 CRL1655 ATCC and is useful for producing a chimeric alphavirus Semliki Forest, which contains a modified sequence of the protein superoxide dismutase B. abortus in their genetic material in the form of RNA.

15. A bacterial strain transformed CHARACTERIZED because it contains the plasmid comprising a chimeric RNA sequence and the protein Cu/Zn SOD protein, where the strain is LMBP 5584.

16. The bacterial strain transformed in accordance with claim 15 CHARACTERIZED because the LMBP 5584 strain is useful as a producer pVSF4.2-SOD plasmid, precursor RNA, which is active in pharmaceutical formulations for ruminants.

17. A chimeric RNA vector according to claims 1 and 2 CHARACTERIZED because the RNA transcribed from plasmid pSFV4.2-SOD has the ability to induce lymphoproliferation of spleen cells of mammals.

18. A chimeric RNA vector according to claims 1 and 2 CHARACTERIZED because both expression systems are capable of inducing a protective immune response against challenge with the B. abortus.

19. An expression system based on Semliki Forest virus CHARACTERIZED because pSFV4.2-SOD expression systems (replicon RNA) and rSFV4.2-SOD, inducing a high level of protection against virulent strain Brucella abortus.

20. Use of a vector RNA from Semliki Forest virus and an exogenous RNA sequence in accordance with claims 1 and 2 CHARACTERIZED because the virus or the chimeric molecule is useful for treating bacterial infections in mammals.

21. Use of a vector of RNA from Semliki Forest virus and an exogenous sequence of RNA in accordance with claims 1 and 2 CHARACTERIZED because the chimeric virus or the chimeric molecule is useful for the preparation of drugs for the treatment of bacterial ruminants.

22. A method for preparing a pharmaceutical formulation in accordance with claims 1 and 2 CHARACTERIZED because it comprises the following steps:
(a) isolation of genes of interest and its promoter sequence from a bacterial strain of B. abortus
(b) cloning of genes of (a) and further cloning of a second system based on an alphavirus vector RNA, which contains the gene for viral replication
(c) incorporation of information (a) and (b) of viral RNA expression system,
(d) development of a construct containing the capsid structural genes, the protein sequence of interest, a cloning vector and expression vector,
(e) development of a construct containing the genes for other structural proteins of interest,
(f) incorporation of information (a) and (e) in a plasmid expression vector,
(g) induction of synthesis of viral particles in a eukaryotic cell,
(h) collecting viral particles from the culture medium through a discontinuous sucrose gradient,
(i) isolate, activate, encapsulated in cationic liposomes,
(j) mixing the liposomes and viral particles with pharmaceutically acceptable excipients.
